(19) United States
(12) Patent Application Publication
(10) Pub. No.: US 2014/0257473 A1
(43) Pub. Date: Sep. 11, 2014

(54) METHODS FOR INHIBITING STENOSIS, OBSTRUCTION, OR CALCIFICATION OF A STENTED HEART VALVE OR BIOPROSTHESIS

(71) Applicant: Nalini Marie Rajamannan, Chicago, IL (US)

(72) Inventor: Nalini Marie Rajamannan, Chicago, IL (US)

(21) Appl. No.: 14/263,438

(22) Filed: Apr. 28, 2014

Related U.S. Application Data
(63) Continuation-in-part of application No. 13/656,925, filed on Oct. 22, 2012.

(51) Int. Cl. A61L 27/54; A61F 2/24

(52) U.S. Cl. CPC A61L 27/54; A61F 2/2412
USPC 623/2.14

(57) ABSTRACT

The present invention relates to methods for inhibiting stenosis, obstruction, or calcification of a valve following implantation of a valve prosthesis which may involve disposing a coating composition on an elastical stent or valve leaflet. The valve prosthesis is mounted on the elastical stent such that the elastical stent is in contact with the valve.

### RELATED APPLICATIONS

This application is a continuation-in-part of U.S. application Ser. No. 13/656,925, filed on Oct. 22, 2012, which is pending, and is also a continuation-in-part of International Patent Appln. Ser. No.: PCT/US13/66142, filed Oct. 22, 2013, which is pending; the entireties of which are hereby incorporated by reference.

### INCORPORATION BY REFERENCE

All documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FIELD OF THE INVENTION

The invention relates to methods for inhibiting stenosis, obstruction, or calcification of heart valves and heart valve prostheses.

### BACKGROUND OF THE INVENTION

The heart is a hollow, muscular organ that circulates blood throughout an organism's body by contracting rhythmically. In mammals, the heart has four-chambers situated such that the right atrium and ventricle are completely separated from the left atrium and ventricle. Normally, blood flows from systemic veins to the right atrium, and then to the right ventricle from which it is driven to the lungs via the pulmonary artery. Upon return from the lungs, the blood enters the left atrium, and then flows to the left ventricle from which it is driven into the systematic arteries.

Four main heart valves prevent the backflow of blood during the rhythmic contractions: the tricuspid, pulmonary, mitral, and aortic valves. The tricuspid valve separates the right atrium and right ventricle, the pulmonary valve separates the right atrium and pulmonary artery, the mitral valve separates the left atrium and left ventricle, and the aortic valve separates the left ventricle and aorta. Generally, patients having an abnormality of a heart valve are characterized as having valvular heart disease.

A heart valve can malfunction either by failing to open properly (stenosis) or by leaking (regurgitation). For example, a patient with a malfunctioning aortic valve can be diagnosed with either aortic valve stenosis or aortic valve regurgitation. In either case, valve replacement by surgical means may be a possible treatment. Replacement valves can be autografts, allografts, or xenografts as well as mechanical valves or valves made partly from valves of other animals, such as pig or cow. Unfortunately, over time, the replacement valves themselves are susceptible to problems such as degeneration, thrombosis, calcification, and/or obstruction. Furthermore, the process of valve replacement may cause perforation in the surrounding tissue, leading also to stenosis, degeneration, thrombosis, calcification, and/or obstruction.

Thus, new methods for inhibiting stenosis, obstruction, or calcification of heart valves and heart valve prostheses are needed.

Citation or identification of any document in this application is not an admission that such document is available as prior art to the present invention.

### SUMMARY OF THE INVENTION

The invention involves methods for inhibiting stenosis, obstruction, or calcification of a valve following implantation of a valve prosthesis in a patient in need thereof, which may comprise: disposing a coating composition on an elastical stent or valve leaflet, wherein the coating composition may comprise one or more therapeutic agents; and securing said valve prosthesis which may comprise a collapsible elastical valve which is mounted on the elastical stent at a desired position in the patient such that the elastical stent is in contact with the valve, thereby inhibiting stenosis, obstruction, or calcification of the valve or stent or surgical placement of a bioprostheses, following implantation of the valve prosthesis in a patient in need thereof.

Accordingly, it is an object of the invention to not encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. §112, first paragraph) or the EPO (Article 83 of the EPC), such that Applicants reserve the right and hereby disclose a disclaimer of any previously described product, process of making the product, or method of using the product.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by the following Detailed Description.

In further aspects, the invention may be set out in the following numbered clauses:

1. A method for inhibiting stenosis, obstruction, or calcification of a bioprosthetic valve following implantation of said bioprosthetic valve in a vessel having a wall, said method comprising:
providing a bioprosthetic valve for surgical replacement of a natural diseased valve, said bioprothestic value including an elastical stent;
providing a coating composition on said elastical stent, bioprosthetic valve or both, wherein the coating composition comprises one or more therapeutic agents;
implanting said bioprosthetic valve into said vessel by surgically removing a natural valve and replacing said natural valve with said bioprosthesis or placing said bioprosthesis over said natural valve having valve leaflets thereby compressing said natural valve leaflets against the vessel wall;
eluting said therapeutic agents from said elastical stent, bioprosthetic valve or both; and
causing the inhibition of stenosis, obstruction, or calcification of the bioprosthesis or the natural valve or both following implantation of the bioprosthetic valve.
   2. The method according to clause 1, wherein the therapeutic agent is selected from paclitaxel, sirolimus, biolimus, everolimus or combinations of the foregoing.
   3. The method according to clause 1, further comprising implanting said bioprosthetic valve by catheterization.
   4. The method according to clause 1, wherein the bioprosthetic valve is an aortic bioprosthetic valve.
   5. The method according to clause 1, wherein the bioprosthetic valve is a bioprosthetic mitral valve.
   6. The method according to clause 1, wherein the bioprosthetic valve is a bioprosthetic pulmonic valve.
   7. The method according to clause 1, wherein the bioprosthetic valve is bioprosthetic tricuspid valve.
   8. The method according to clause 1, wherein the bioprosthetic valve comprises one or more cusps of biological origin.
   9. The method according to clause 8, wherein the one or more cusps is porcine, bovine, or human.
   10. The method according to clause 8, further comprising introducing a nucleic acid encoating a nitric oxide synthase into the one or more cusps.
   11. The method according the clause 8, further comprising introducing a drug eluting treating encoating the one or more cusps on both sides with an anti-proliferative and anti-calcification treatment.
   12. The method according to clause 1, wherein the elastical stent is substantially cylindrical.
   13. The method according to clause 12, wherein the diameter of the elastical stent is about 15 mm to about 42 mm.
   14. A valve prosthesis comprising:
an elastical stent;
a bioprosthetic valve having leaflets operably coupled to said elastical stent;
a therapeutic agent on the elastical stent, bioprosthetic valve on sides of the leaflets or both, said therapeutic agent structured to elute from said elastical stent, bioprosthetic valve leaflets or both;
wherein said bioprosthetic valve is structured to be implanted in vessel having a vessel wall to replace a natural diseased valve;
said bioprosthetic valve structured to inhibit stenosis, obstruction, or calcification of the bioprosthetic valve and natural valve following implantation of the bioprosthetic valve prosthesis.
   15. The bioprosthetic valve of clause 14 wherein said bioprosthetic valve is coupled to a surgical sewing ring.
   16. The bioprosthetic valve of clause 14, wherein the therapeutic agent is selected from paclitaxel, sirolimus, biolimus, everolimus, zotarolimus and combinations of the foregoing.
   17. The bioprosthetic valve of clause 14, said valve sized and constructed to be implanted by catherization in a coronary valve of the patient.
   18. The bioprosthetic valve of clause 14, wherein the valve is an aortic valve.
   19. The bioprosthetic valve of clause 14, wherein the bioprosthetic valve comprises one or more cusps of biological origin.
   20. The bioprosthetic valve of clause 19, wherein the one or more cusps are porcine, bovine, or human.
   21. The bioprosthetic valve of clause 19 further comprising a nucleic acid encoating a nitric oxide synthases into one or more of the cusps.
   22. The elastical stent of clause 14 sized, constructed and arranged in a substantially cylindrical configuration.
   23. The valve prosthesis of clause 14 wherein a diameter of the elastical stent is about 15 mm to about 42 mm.
   24. The method of clause 1 wherein the elastical stent has a surface constructed and arranged to reduce neointimal proliferation.
   25. The valve prosthesis of clause 14 wherein the elastical stent has a surface constructed and arranged to reduce neointimal proliferation.
   26. The method of clause 1 wherein the bioprosthetic valve has a surface constructed and arranged to reduce neointimal proliferation and calcification.
   27. The valve prosthesis of clause 14, wherein the therapeutic agent is selected from paclitaxel, sirolimus, biolimus, everolimus, zotarolimus and combinations of the foregoing.
   28. The bioprosthetic valve of clause 14, said valve is sized constructed and arranged to be implanted by catherization in a coronary valve of the patient.
   29. The bioprosthetic valve of clause 14, wherein the valve is an aortic valve.
   30. The bioprosthetic valve of clause 14, wherein the bioprosthetic valve comprises one or more cusps of biological origin.
   31. The bioprosthetic valve of clause 30, wherein the one or more cusps are porcine, bovine, or human.
   32. The bioprosthetic valve of clause 14 and further comprising a nucleic acid encoating a nitric oxide synthases into one or more of the cusps.
   33. The method of clause 1 further comprising providing a sewing ring operably coupled to said bioprosthetic valve, said sewing ring having a surface constructed and arranged to reduce neointimal proliferation.
   34. The valve prosthesis of clause 14 further comprising a sewing ring operably coupled to said bioprosthetic valve, said sewing ring having a surface constructed and arranged to reduce neointimal proliferation and pannus formation.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1 of the drawings is a front perspective view of a bioprosthetic aortic valve showing leaflet 16 and stent 10.

FIG. 2 of the drawings is a front perspective view of another type of aortic valve showing the leaflets 26 and stent 28.

FIG. 3 of the drawings is a front schematic view showing an aorta with the aortic valve of FIG. 1 inserted therein at the time of initial implantation before any disease can develop in the aorta from the stent.

FIG. 4 of the drawings is a front cut-away view of an aorta 32 showing the aortic valve of FIG. 2 inserted therein at the time of initial implantation before any disease can develop in the aorta 32 from the stent 28.

FIG. 5 of the drawings is a schematic view showing an aorta having the aortic valve of FIG. 1 therein, in which the aorta surrounding the stent has been partially blocked by stenosis secondary to vascular smooth muscle cell proliferation and differentiation to bone forming cells after injury from the stent adjacent to the aorta, and c-kit stem cell proliferation and differentiation to bone formation cells secondary to inflammation and homing of c-kit stem cells to become bone forming cells.

FIG. 6 of the drawings is a front cut-away view of an aorta showing the aorta surrounding the stent of FIG. 2 partially blocked by stenosis secondary to vascular smooth muscle cell proliferation and differentiation to bone forming cells after injury from the stent adjacent to the aorta, and c-kit stem cell proliferation and differentiation to bone formation cells secondary to inflammation and homing of c-kit stem cells to become bone forming cells.

FIG. 7 of the drawings is a top view of the mesh utilized in the stented aortic valve of FIG. 2 of the drawings.

FIG. 8 of the drawings is a top view showing the mesh of FIG. 7 coated with an anti-proliferative coating to prevent stenosis in the stent surrounding the aorta to prevent the smooth muscle cell proliferation and calcification in the aorta, this is the treatment and the invention for this type of stent.

FIG. 9 of the drawings is a photograph of insertion of the Portico™ valve prosthesis into the aortic artery of the patient using a catheter.

FIG. 10 is a photograph showing a Portico™ transcatheter heart valve and an 18-F delivery catheter for insertion of the heart valve into the aorta.

FIG. 11 is a drawing showing the treatment of the valve leaflet of FIG. 1 with an anti-proliferative coating along the stent 10 and the valve leaflet 16.

FIG. 12 is a drawing showing the treatment of the valve leaflet of FIG. 2 with an anti-proliferative coating along the stent and the valve leaflet.

### DETAILED DESCRIPTION OF THE INVENTION

The invention provides a method for inhibiting stenosis, obstruction, or calcification of a stented aorta and valve leaflet or bioprosthesis with or without a sewing ring, following implantation of a valve prosthesis in a patient in need thereof, which may comprise: disposing a coating composition on an elastical stent, wherein the coating composition may comprise one or more therapeutic agents; securing a bioprosthetic collapsible elastical valve which is mounted on the elastical stent at a desired position in the patient such that the elastical stent is in contact with a natural valve that may or may not have been surgically removed, and optionally disposing a coating composition on both sides of the valve leaflets, the stent or a sewing ring to which the bioprosthetic valve is secured thereby inhibiting stenosis, obstruction, or calcification of the stented aorta following implantation of the stented valve prosthesis in a patient in need thereof or the surgical replacement of a bioprothesis that replaces a native valve.

As used herein, the term “stenosis” may refer to the narrowing of a heart valve that could block or obstruct blood flow from the heart and cause a back-up of flow and pressure in the heart. Valve stenosis may result from various causes, including, but not limited to, scarring due to disease, such as rheumatic fever; progressive calcification; progressive wear and tear; among others. This is important not for the stented treatment but for the valve—is this flowing well with the rest of the patent.

As used herein, the term “valve” may refer to any of the four main heart valves that prevent the backflow of blood during the rhythmic contractions. The four main heart valves are the tricuspid, pulmonary, mitral, and aortic valves. The tricuspid valve separates the right atrium and right ventricle, the pulmonary valve separates the right atrium and pulmonary artery, the mitral valve separates the left atrium and left ventricle, and the aortic valve separates the left ventricle and aorta.

In an embodiment of the method, the bioprosthetic valve and the diseased valve may be an aortic valve, pulmonary valve, tricuspid valve, or mitral valve.

As used herein, the term “valve prosthesis” may refer to a device used to replace or supplement a heart valve that is defective, malfunctioning, or missing. Examples of valve prostheses include, but are not limited to, bioprostheses; mechanical prostheses, and the like including, ATS 3fs® Aortic Bioprosthesis, Carpentier-Edwards PERIMOUNT Magna Ease Aortic Heart Valve, Carpentier-Edwards PERIMOUNT Magna Aortic Heart Valve, Carpentier-Edwards PERIMOUNT Magna Mitral Heart Valve, Carpentier-Edwards PERIMOUNT Aortic Heart Valve, Carpentier-Edwards PERIMOUNT Plus Mitral Heart Valve, Carpentier-Edwards PERIMOUNT Theon Aortic Heart Valve, Carpentier-Edwards PERIMOUNT Theon Mitral Replacement System, Carpentier-Edwards Aortic Porcine Bioprosthesis, Carpentier-Edwards Duraflex Low Pressure Porcine Mitral Bioprosthesis, Carpentier-Edwards Duraflex mitral bioprosthesis (porcine), Carpentier-Edwards Mitral Porcine Bioprosthesis, Carpentier-Edwards S.A.V. Aortic Porcine Bioprosthesis, Edwards Prima Plus Stentless Bioprosthesis, Edwards Sapien Transcatheter Heart Valve, Medtronic, Freestyle® Aortic Root Bioprosthesis, Hancock® II Stented Bioprosthesis, Hancock II Ultra® Bioprosthesis, Mosaic® Bioprosthesic, Mosaic Ultra® Bioprosthesis, St. Jude Medical, Biocor®, Biocor™ Supra, Biocor® Pericardia, Biocor™ Stentless, Epic™, Epic Supra™, Toronto Stentless Porcine Valve (SPV®), Toronto SPV II®, Trifecta, Sorin Group, Mitroflow Aortic Pericardial Valve®, Cryolife, Cryolife aortic Valve® Cryolife pulmonic Valve®, Cryolife-O'Brien stentless aortic xenograft Valve®

Generally, bioprostheses comprise a valve having one or more cusps and the valve is mounted on a frame or stent, both of which are typically elastical. As used herein, the term “elastical” means that the device is capable of flexing, collapsing, expanding, or a combination thereof. The cusps of the valve are generally made from tissue of mammals such as, without limitation, pigs (porcine), cows (bovine), horses, sheep, goats, monkeys, and humans.

According to the method of the present invention, the valve may be a collapsible elastical valve having one or more cusps and the collapsible elastical valve may be mounted on an elastical stent.

In an embodiment, the collapsible elastical valve may comprise one or more cusps of biological origin.

In another embodiment, the one or more cusps are porcine, bovine, or human.

Examples of bioprostheses may comprise a collapsible elastical valve having one or more cusps and the collapsible elastical valve is mounted on an elastical stent include, but are not limited to, the SAPIEN transcatheter heart valve manufactured Edwards Lifesciences, and the CoreValve® transcatheter heart valve manufactured by Medtronic and Portico-Melody by Medtronic.

The elastical stent portion of the valve prosthesis used in the present invention may be self-expandable or expandable by way of a balloon catheter. The elastical stent may comprise any biocompatible material known to those of ordinary skill in the art. Examples of biocompatible materials include, but are not limited to, ceramics; polymers; stainless steel; titanium; nickel-titanium alloy, such as Nitinol; tantalum; alloys containing cobalt, such as Elgiloy® and Phynox®; and the like.

According to the method of the present invention, a coating composition which may comprise one or more therapeutic agents is disposed on the elastical stent portion of the valve prosthesis. The process of disposing the coating composition which may comprise one or more therapeutic agents may be any process known in the art. The coating compositions may be prepared by dissolving or suspending a polymer and therapeutic agent in a solvent. Suitable solvents that may be used to prepare the coating compositions include those that may dissolve or suspend the polymer and therapeutic agent in solution. Examples of suitable solvents include, but are not limited to, tetrahydrofuran, methylethylketone (MEK), chloroform, toluene, acetone, isooctane, 1,1,1, trichloroethane, dichloromethane, isopropanol, and mixtures thereof. However, solvents are not required in many cases.

The coating compositions may be applied by any method to the surface of the elastical stent portion of the valve prosthesis or bioprostheses and sewing ring, known by one skilled in the art. Suitable methods for applying the coating compositions to the surface of the elastical stent portion of the valve prosthesis include, but are not limited to, spray-coating, painting, rolling, electrostatic deposition, ink jet coating, and a batch process such as air suspension, pan-coating or ultrasonic mist spraying, or a combination thereof.

After the coating composition has been applied, it may be cured. As used herein, “curing” may refer to the process of converting any polymeric material into the finished or useful state by the application of heat, vacuum, and/or chemical agents, which application induces physico-chemical changes. The applicable time and temperature for curing are determined by the particular polymer involved and particular therapeutic agent used as known by one skilled in the art. Also, after the elastical stent is coated, it may be sterilized by methods of sterilization as known in the art (see, e.g., Guidance for Industry and FDA Staff—Non-Clinical Engineering Tests and Recommended Labeling for Intravascular Stents and Associated Delivery Systems http://www.fda.gov/medicaldevices/deviceregulationandguidance/guidancedocuments/ucm071863.htm and U.S. Pat. No. 7,998,404 entitled “Reduced temperature sterilization of stents.”

As used herein, the term “therapeutic agent” may refer to biologically active materials, The therapeutic agents named herein include their analogues and derivatives. Suitable therapeutic agents include, but are not limited to, microtubule stabilizing agents, such as paclitaxel, its analogues, and its derivatives; macrolide antibiotic agents, such as sirolimus (rapamycin) its analogues, and its derivatives; or combinations thereof. Bioliums or Everolimus Biolimus (see “Transcatheter Aortic Valve Replacement with St. Jude Medical Portico Valve”, Journal of American College of Cardiology, Vol. 60, No. 7, 2012:581-6, 6 pages, dated Aug. 14, 2012 which is hereby incorporated by reference.) The elastical stent portion of the valve prosthesis may be made to provide a desired release profile of the therapeutic agent.

There are also several other anti-proliferative drugs under investigation in human clinical trials. In general, these are analogues of sirolimus. Like sirolimus, these block the action of mTOR. Medtronic has developed zotarolimus; unlike sirolimus and paclitaxel, this sirolimus analogue designed for use in stents with phosphorylcholine as a carrier. Their ZoMaxx stent is a zotarolimus-eluting, stainless steel and tantalum-based stent; a modified phosphorylcholine slowly releases the zotarolimus. Zotarolimus has been licensed to Medtronic which is researching the effectiveness in a drug-eluting stent of their own. The Medtronic Endeavor stent, which is a cobalt alloy, also uses phosphorylcholine to carry the zotarolimus was approved for use in Europe in 2005 is now close to U.S. FDA approval.

http://en.wikipedia.org/wiki/Drug-eluting_stent discloses a listing of current FDA approved and investigational drugs undergoing testing for treatment of stents.

Clinical trials are currently examining two stents carrying everolimus, an analog of sirolimus. Guidant, which has the exclusive license to use everolimus in drug-eluting stents, is the manufacturer of both stents. The Guidant vascular business was subsequently sold to Abbott. The Champion stent uses a bioabsorbable polylactic acid carrier on a stainless steel stent. In contrast, its Xience stent uses a durable (non-bioabsorbable) polymer on a cobalt alloy stent.

One alternative to drug-eluting stents is a stent surface constructed and arranged to reduce the neointimal proliferation. One such is the Genous bioengineered stent.

In place of the stainless steel (and now cobalt chrome) currently used in stents, various biodegradable frameworks are under early phases of investigation. Since metal, as a foreign substance, provokes inflammation, scarring, and thrombosis (clotting), it is hoped that biodegradable or bioabsorbable stents may prevent some of these effects. A magnesium alloy—based stent has been tested in animals, though there is currently no carrier for drug elution. A promising biodegradable framework is made from poly-L-lactide, a polymer of a derivative of L-lactic acid. One of these stents, the Igaki-Tamai stent, has been studied in pigs; tranilast and paclitaxel have been used as eluted drugs. Again none of these have been used suggested or implied for use in the aorta.

According to the method of the present invention, the valve prosthesis may be secured at a desired position in the heart of a patient such that the elastical stent is in contact with the valve or the walls of the valve. The desired position of the valve prosthesis may be easily determined using methods known to those of ordinary skill in heart valve replacement echo imaging, CT imaging, catherization. In an embodiment, the valve prosthesis may be configured to be implanted by way of cardiac catheterization echo imaging, CT imaging, catherization. Catheter delivery of the valve prosthesis may be accomplished using methods well known to those skilled in the art, such as mounting the elastical stent portion on an inflatable balloon disposed at the distal end of a delivery catheter and expanding the valve prosthesis at the desired position.

The elastical stent portion of the valve prosthesis may be any shape cylindrical (final shape is cylinder may be funnel shaped original all required to contact the valve or walls of the valve where, without being bound to theory, the therapeutic agents are released and absorbed by the valve or walls of the valve, or the aorta including aortic valve, mitral valve, tricuspid valve, vena cava valve.

In an embodiment, the elastical stent portion may be substantially cylindrical so as to be able to contact the valve or walls of the valve upon securing.

In another embodiment, the diameter of the elastical stent portion may be about 15 mm to about 42 mm.

According to an embodiment of the present invention, the method further may comprise introducing a nucleic acid encoding a nitric oxide synthase into the one or more cusps of the valve prosthesis. Methods for introducing a nucleic acid encoding a nitric oxide synthase into the one or more cusps are described in U.S. Pat. No. 6,660,260, issued Dec. 9, 2003, and is hereby incorporated by reference in its entirety.

Although the present invention and its advantages have been described in detail, it should be understood that various changes, substitutions and alterations can be made herein without departing from the spirit and scope of the invention as defined in the appended claims. There is no method of treatment in the stents in their current form to prevent the stenosis that can develop from the stent injury to the aorta. This is the invention as described in this application to prevent stenosis that can develop in the vascular aorta from these stented valve therapy or in the valve leaflets attached to the stented valve.

The present invention will be further illustrated in the following Examples which are given for illustration purposes only and are not intended to limit the invention in any way.

### EXAMPLES

For coronary stents see “Effect of Biolimus-Eluting Stents with Biodegradable Polymer vs. Bare-Metal Stents on Cardiovascular Events Among Patients with Acute Myocardial Infarction, The COMFORTABLE AMI Randomized Trial”, JAMA: The Journal of the American Medical Association, Vol. 308, No. 8, Aug. 22/29, 2012 is hereby incorporated by reference.

Having thus described in detail preferred embodiments of the present invention, it is to be understood that the invention defined by the above paragraphs is not to be limited to particular details set forth in the above description, as many apparent variations thereof are possible without departing from the spirit or scope of the present invention.

### DETAILED DESCRIPTION OF THE PREFERRED EMBODIMENT

As best seen in FIG. 1 of the drawings, an elastical stent 10 having a coating 12 disposed thereon, said coating composition comprising one or more therapeutic agents. The method comprises the steps of disposing the coating composition 12 on the elastical stent 10. A valve prosthesis 14 is mounted on the elastical stent 10. The valve prosthesis 14 is a collapsible elastical valve 16 which is mounted on the elastical stent 10 at a desired position 18 in the patient. The stent and valve are positioned within a coronary valve artery the aorta. The elastical stent 10 is in contact with the valve 16. The coating composition 12 inhibits stenosis, obstruction or calcification of the valve prosthesis 16 along with implantation of the valve prosthesis 14 in the patient.

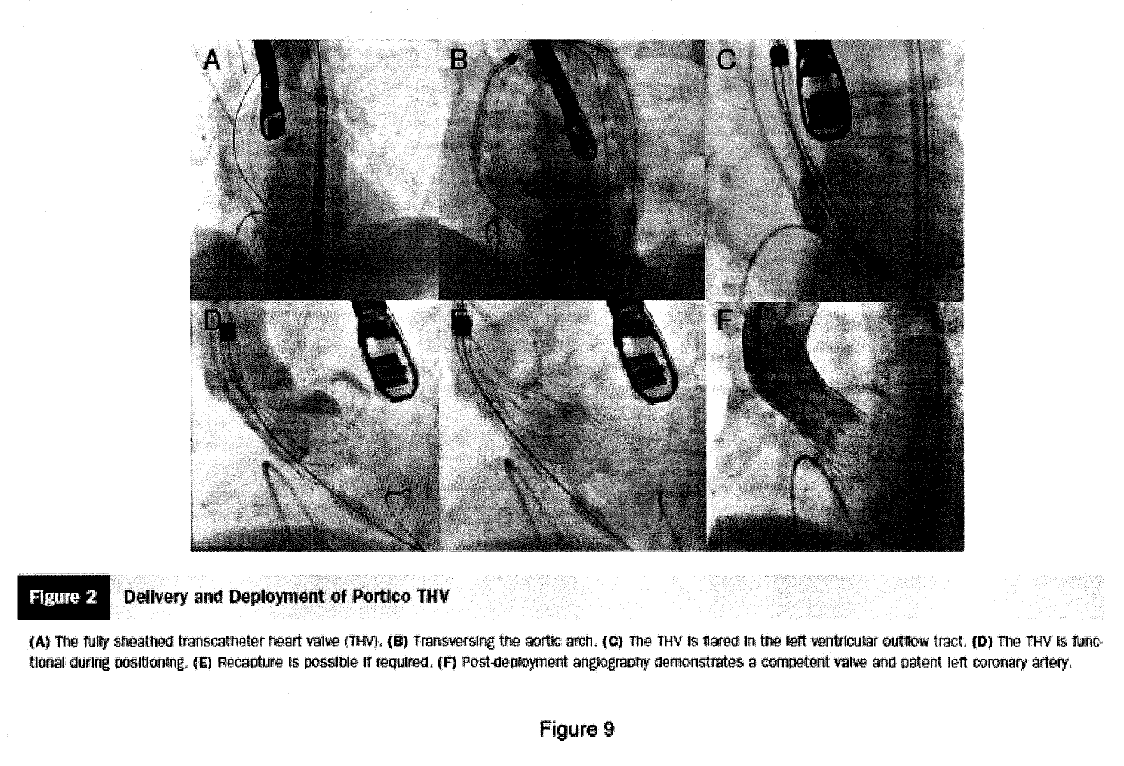

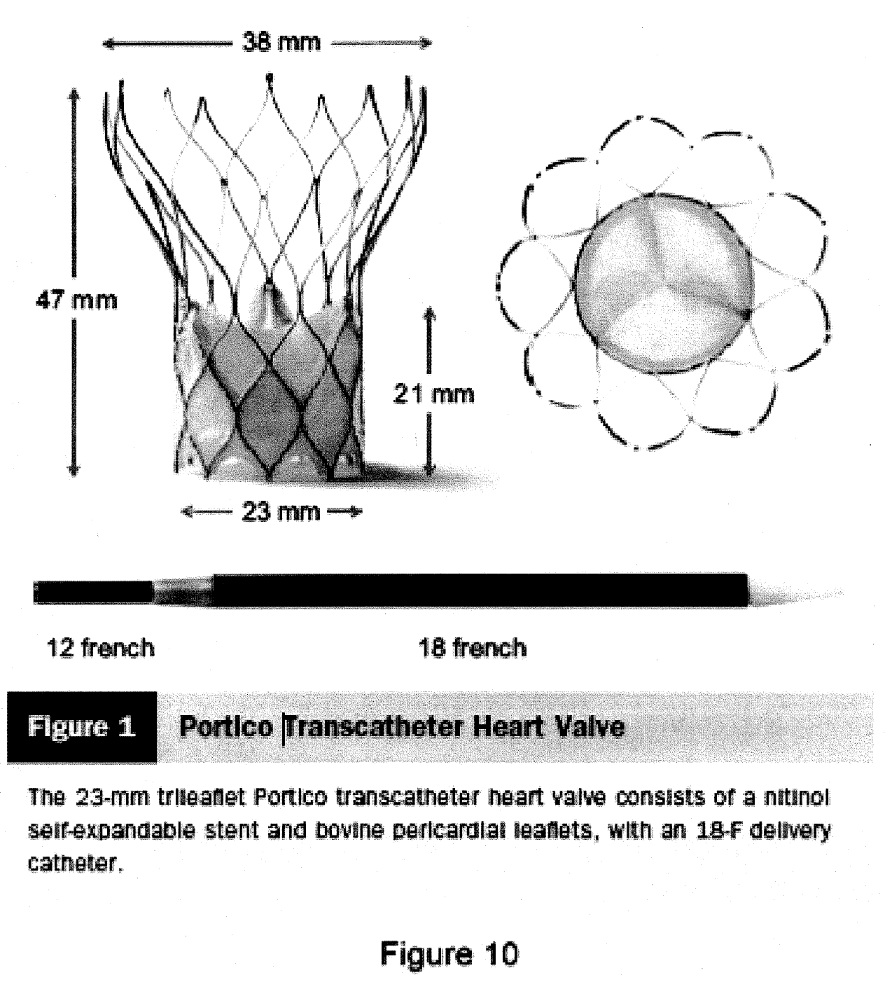

The therapeutic agent referred to above may be selected from the group comprising paclitaxel, sirolimus, biolimus, and everolimus. Implantation of the valve is preferably performed using a catheter, as shown in the attached article from the Journal of the of the American College of Cardiology, Vol. 60, No. 7, 2012, Aug. 14, 2012; 581-6 FIG. 9, Transcatheter Aortic Valve Replacement with the St Jude Medical Portico Valve which is hereby incorporated by reference. An aorta 20 is shown in FIG. 3 of the drawings with, a valve prosthesis 14 inserted therein. In one or more alternative embodiments of the invention the collapsible elastical valve 16 may have one or more cusps 22 of biological origin. The cusp 22 may be porcine, bovine, or human as is commonly known in the art. In an additional alternative embodiment of the invention nucleic acid 24 encoding a nitric oxide synthase may be introduced into one or more of the cusps 22 to inhibit stenosis, obstruction or calcification of the valve. In a preferred embodiment the elastical stent 10 is substantially cylindrical and is from approximately 18 millimeters to about 29 millimeters in length. Although prevention of stenosis, calcification and obstruction of other valves other than the aorta was previously known it was not expected that an anti-proliferative agent would be required because of the size of the aorta, which is bigger than coronary valves. The aorta is 2 centimeters in diameter whereas coronary arteries are 4 millimeters in diameter. Most stents such as that shown in FIGS. 1-8 are constructed of titanium so as to avoid thrombosis. It has been previously known to utilize statins for coronary valves to prevent stenosis, obstruction or calcification but not with aortic valves. The statin utilized is 80 milligrams of Lipitor a day.

In a preferred embodiment the coating 12 on elastic stent 10 is paclitaxel, which is a mitotic inhibitor, previously used in cancer chemotherapy. It previously was sold as dissolved in cremafor EL and etheynol as a delivery agent. A newer formulation has paclitaxel bound to albumin sold under the trademark Abraxane. It is known to use paclitaxel to prevent restenosis. Paclitaxel is used as an anti-proliferative agent for the prevention of restenosis (recurring narrowing) of coronary stents locally delivered to the wall of the coronary artery. A paclitaxel coating limits the growth of neointima (scar tissue) within stents. The article Paclitaxel footnote 39. Paclitaxel stent coating inhibits neointima hyperplasia at four weeks and a poor sign model of coronary restenosis PMID 11342479. “Paclitaxel”, Wikipedia, the free encyclopedia, http://en.wikipedia.org/wiki/Paclitaxel, Oct. 4, 2012 is hereby incorporated by reference. In an alternative embodiment biolimus, and equipotent sirolimus analog from biodegradable polylactic acid was not inferior and potentially better than sirolimus eluding stents in terms of major adverse clinical events, in a large clinical trial with follow-up of four years.

As noted above the elastical stent 10 shown in FIGS. 1-6 may comprise any biocompatible material known to those of ordinary skill in the art. Examples of bio compatible materials include but are not limited to ceramics; polymers; stainless steel, titanium; nickel-titanium alloy such as Nitinol; tantalum; alloys containing cobalt such as elgioloy and Finox® and the like.

As best seen in FIG. 8, according to the method of the present invention, a coating composition 12 which may comprise one or more therapeutic agents is disposed on the elastical stent 10 portion of the valve prosthesis 14. The process of disposing the coating composition 12, which may comprise one or more therapeutic agents, may be any process known in the art. The coating compositions 12 may be prepared by dissolving or suspending a polymer and therapeutic agent in a solvent. Suitable solvents that may be used to prepare the coating compositions 12 include those that may dissolve or suspend the polymer and therapeutic agent in solution. Examples of suitable solvents include, but are not limited to, tetrahydrofuran, methylethylketone (MEK), chloroform, toluene, acetone, isooctane, 1,1,1, trichloroethane, dichloromethane, isopropanol, and mixtures thereof.

The coating compositions 12 may be applied by any method to the surface of the elastical stent 10 portion of the valve prosthesis 14 known by one skilled in the art. Suitable methods for applying the coating compositions 12 to the surface of the elastical stent 10 portion of the valve prosthesis 14 include, but are not limited to, spray-coating, painting, rolling, electrostatic deposition, ink jet coating, and a batch process such as air suspension, pan-coating or ultrasonic mist spraying, or a combination thereof.

After the coating composition 12 has been applied, it may be cured. As used herein, “curing” may refer to the process of converting any polymeric material into the finished or useful state by the application of heat, vacuum, and/or chemical agents, which application induces physico-chemical changes. The applicable time and temperature for curing are determined by the particular polymer involved and particular therapeutic agent used as known by one skilled in the art. Also, after the elastical stent is coated, it may be sterilized by methods of sterilization as known in the art (see, e.g., Guidance for Industry and FDA Staff—Non-Clinical Engineering Tests and Recommended Labeling for Intravascular Stents and Associated Delivery Systems http://www.fda.gov/medicaldevices/deviceregulationandguidance/guidancedocuments/ucm071863.htm and U.S. Pat. No. 7,998,404 entitled “Reduced temperature sterilization of stents.”

As used herein, the term “therapeutic agent” may refer to biologically active materials. The therapeutic agents named herein include their analogues and derivatives. Suitable therapeutic agents include, but are not limited to, microtubule stabilizing agents, such as paclitaxel, its analogues, and its derivatives; macrolide antibiotic agents, such as sirolimus (rapamycin) its analogues, and its derivatives; or combinations thereof. Bioliums or Everolimus Biolimus (see “Transcatheter Aortic Valve Replacement with St. Jude Medical Portico Valve”, Journal of American College of Cardiology, Vol. 60, No. 7, 2012:581-6, 6 pages, dated Aug. 14, 2012 which is hereby incorporated by reference. The elastical stent portion of the valve prosthesis may be made to provide a desired release profile of the therapeutic agent.

Implantation of a Portico trans catheter heart valve may be seen in FIG. 10, attached. In a preferred embodiment the heart valve 16 transverses the aortic arch 24. The trans catheter heart valve is flared in a left ventricular outflow tract 26. The trans catheter heart valve is in functional during positioning. The valve when open may be seen in FIG. 1. Transcatheter aortic valve replacement with the St. Jude Medical Portico valve: first-in-human experience. Willson A B, Rodes-Cabau J, Wood D A, Leipsic J, Cheung A, Toggweiler S, Binder R K, Freeman M, DeLarochelliere R, Moss R, Nombela-Franco L, Dumont E, Szummer K, Fontana G P, Makkar R, Webb J G. J. Am Coll Cardiol. 2012 Aug. 14; 60(7):581-6. Epub 2012 May 30. Willson A B, Rodes-Cabau J, Wood D A, Leipsic J, Cheung A, Toggweiler S, Binder R K, Freeman M, DeLarochelliere R, Moss R, Nombela-Franco L, Dumont E, Szummer K, Fontana G P, Makkar R, Webb J G. J. Am Coll Cardiol. 2012 Aug. 14; 60(7):581-6. Epub 2012 May 30.

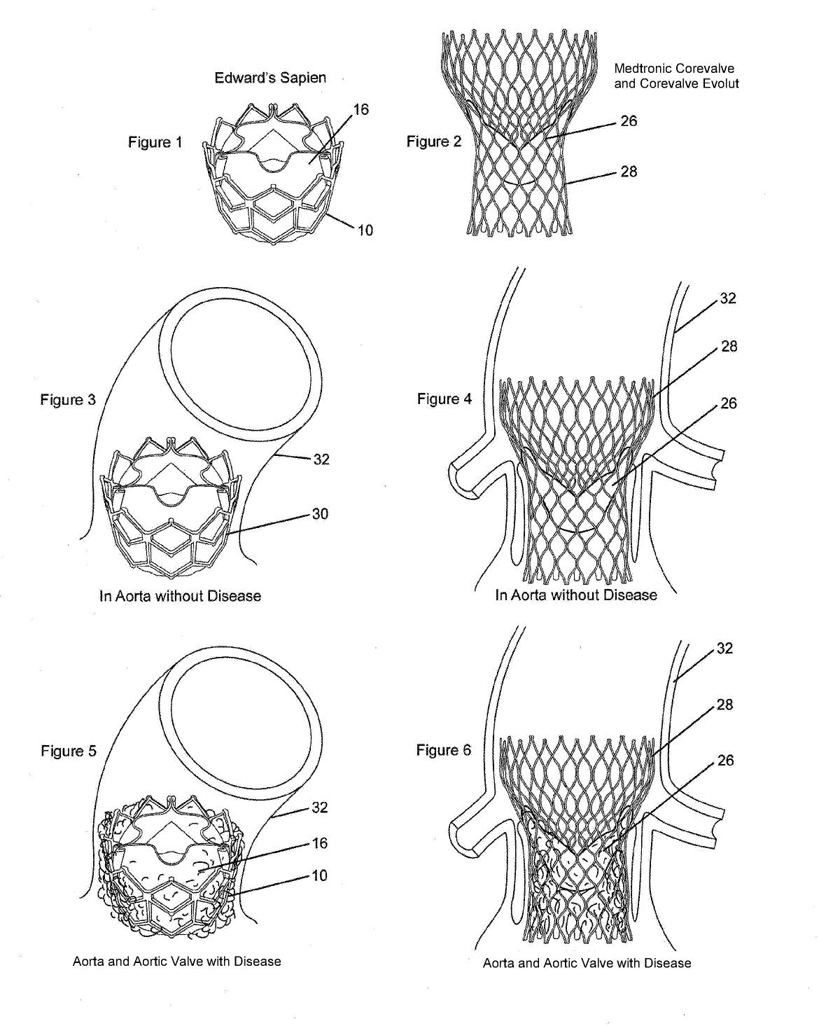

As seen in FIG. 2 the Medtronic core valve 26 is disclosed within a coated stent 28. The Edwards LifeSciences Sapien Valve, New techniques for the treatment of valvular aortic stenosis—transcatheter aortic valve implantation with the SAPIEN heart valve. Thielmann M, Eggebrecht H, Wendt D, Kahlert P, Ideler B, Kottenberg-Assenmacher E, Erbel R, Jakob H. Minim Invasive Ther Allied Technol. 2009; 18(3):131-4. Geometry and degree of apposition of the CoreValve ReValving system with multislice computed tomography after implantation in patients with aortic stenosis. Schultz C J, Weustink A, Piazza N, Often A, Mollet N, Krestin G, van Geuns R J, de Feyter P, Serruys P W, de Jaegere P. J Am Coll Cardiol. 2009 Sep. 1; 54(10):911-8.

As seen in FIG. 3, an Edwards Sapien valve 30 is disclosed contained within an aorta 32, which is without disease. Similarly, in FIG. 4, the Medtronic Core Valve 26 within stent 28 is positioned with aorta 32. Both in FIGS. 3 and 4 the aortas are shown without disease. As best seen in FIG. 5 of the drawings aorta 32 has the Edwards Sapien valve 30, also numbered 16, is fixedly positioned within stent 10. However, in this case the stent and valve are not coated with the present invention's anti proliferative agent. Accordingly, stenosis, obstruction and calcification have occurred. Similarly, in FIG. 6, the Medtronic core valve 26 positioned within stent 28 is contained within aorta 32. Again, without the anti-proliferative agents of the present invention stenosis, obstruction, and calcification have occurred. The leaflets or cusps 22 shown in FIGS. 1-6 may be constructed of tissue from mammal. The stents may be constructed also of biodegradable polymers providing controlled drug release or alternatively biological leaflets can be porcine or human cells. Alternatively, any non murine species having heart valve tissue including, without limitation, mammals such as pigs, cows, horses, sheep, goats, monkeys, and humans can be utilized for the leaflets. U.S. Pat. No. 6,660,260 which describes such heart valve cells is hereby incorporated by reference.

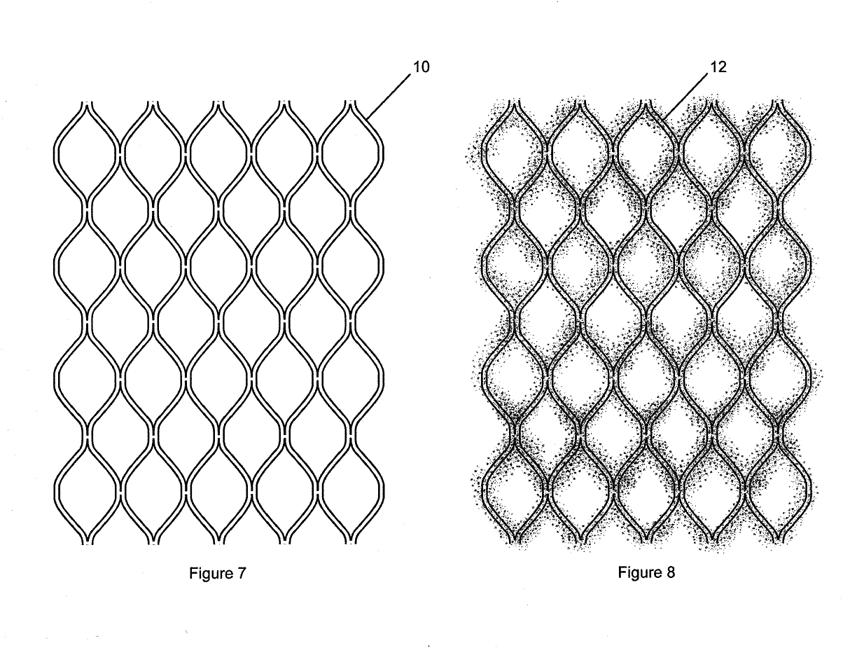

As seen in FIGS. 7 and 8, a stent 10 is disclosed without any coating 12 thereon. In FIG. 8, the same stent 10 is shown having a coating 12 thereon which is an anti-proliferative agent such as paclitaxel, sirolimis, everolimus or biolimius.

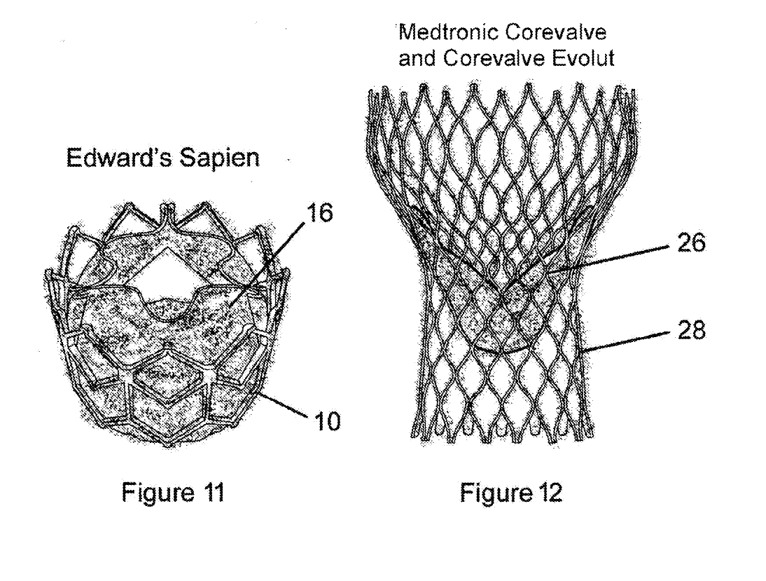

FIG. 11 and FIG. 12 are the treated valves and stents is shown having a coating 12 thereon which is an anti-proliferative agent such as paclitaxel, sirolimis, everolimus or biolimius.

While the present invention is shown with several specific embodiments, the invention is not limited thereto, except in so far as those who have the disclose before them are able to make modifications and variations therein without departing from the scope of the invention.

What is claimed is:

1. A method for inhibiting stenosis, obstruction, or calcification of a bioprosthetic valve following implantation of said bioprosthetic valve in a vessel having a wall, said method comprising:
providing a bioprosthetic valve for surgical replacement of a natural diseased valve, said bioprothestic value including an elastical stent;
providing a coating composition on said elastical stent, bioprosthetic valve or both, wherein the coating composition comprises one or more therapeutic agents;
implanting said bioprosthetic valve into said vessel by surgically removing a natural valve and replacing said natural valve with said bioprosthesis or placing said bioprosthesis over said natural valve having valve leaflets thereby compressing said natural valve leaflets against the vessel wall;
eluting said therapeutic agents from said elastical stent, bioprosthetic valve or both; and
causing the inhibition of stenosis, obstruction, or calcification of the bioprosthesis or the natural valve or both following implantation of the bioprosthetic valve.

2. The method according to claim 1, wherein the therapeutic agent is selected from paclitaxel, sirolimus, biolimus, everolimus or combinations of the foregoing.

3. The method according to claim 1, further comprising implanting said bioprosthetic valve by catheterization.

4. The method according to claim 1, wherein the bioprosthetic valve is an aortic bioprosthetic valve.

5. The method according to claim 1, wherein the bioprosthetic valve is a bioprosthetic mitral valve.

6. The method according to claim 1, wherein the bioprosthetic valve is a bioprosthetic pulmonic valve.

7. The method according to claim 1, wherein the bioprosthetic valve is bioprosthetic tricuspid valve.

8. The method according to claim 1, wherein the bioprosthetic valve comprises one or more cusps of biological origin.

9. The method according to claim 8, wherein the one or more cusps is porcine, bovine, or human.

10. The method according to claim 8, further comprising introducing a nucleic acid encoating a nitric oxide synthase into the one or more cusps.

11. The method according the claim 8, further comprising introducing a drug eluting treating encoating the one or more cusps on both sides with an anti-proliferative and anti-calcification treatment.

12. The method according to claim 1, wherein the elastical stent is substantially cylindrical.

13. The method according to clause 12, wherein the diameter of the elastical stent is about 15 mm to about 42 mm.

14. A valve prosthesis comprising:
an elastical stent;
a bioprosthetic valve having leaflets operably coupled to said elastical stent;
a therapeutic agent on the elastical stent, bioprosthetic valve on sides of the leaflets or both, said therapeutic agent structured to elute from said elastical stent, bioprosthetic valve leaflets or both;
wherein said bioprosthetic valve is structured to be implanted in vessel having a vessel wall to replace a natural diseased valve;
said bioprosthetic valve structured to inhibit stenosis, obstruction, or calcification of the bioprosthetic valve and natural valve following implantation of the bioprosthetic valve prosthesis.

15. The bioprosthetic valve of claim 14 wherein said bioprosthetic valve is coupled to a surgical sewing ring.

16. The bioprosthetic valve of claim 14, wherein the therapeutic agent is selected from paclitaxel, sirolimus, biolimus, everolimus, zotarolimus and combinations of the foregoing.

17. The bioprosthetic valve of claim 14, said valve sized and constructed to be implanted by catherization in a coronary valve of the patient.

18. The bioprosthetic valve of claim 14, wherein the valve is an aortic valve.

19. The bioprosthetic valve of claim 14, wherein the bioprosthetic valve comprises one or more cusps of biological origin.

20. The bioprosthetic valve of claim 19, wherein the one or more cusps are porcine, bovine, or human.

21. The bioprosthetic valve of claim 19 further comprising a nucleic acid encoating a nitric oxide synthases into one or more of the cusps.

22. The elastical stent of claim 14 sized, constructed and arranged in a substantially cylindrical configuration.

23. The valve prosthesis of clause 14 wherein a diameter of the elastical stent is about 15 mm to about 42 mm.

24. The method of claim 1 wherein the elastical stent has a surface constructed and arranged to reduce neointimal proliferation.

25. The valve prosthesis of claim 14 wherein the elastical stent has a surface constructed and arranged to reduce neointimal proliferation.

26. The method of claim 1 wherein the bioprosthetic valve has a surface constructed and arranged to reduce neointimal proliferation and calcification.

27. The valve prosthesis of claim 14, wherein the therapeutic agent is selected from paclitaxel, sirolimus, biolimus, everolimus, zotarolimus and combinations of the foregoing.

28. The bioprosthetic valve of claim 14, said valve is sized constructed and arranged to be implanted by catherization in a coronary valve of the patient.

29. The bioprosthetic valve of claim 14, wherein the valve is an aortic valve.

30. The bioprosthetic valve of claim 14, wherein the bioprosthetic valve comprises one or more cusps of biological origin.

31. The bioprosthetic valve of claim 30, wherein the one or more cusps are porcine, bovine, or human.

32. The bioprosthetic valve of claim 14 and further comprising a nucleic acid encoating a nitric oxide synthases into one or more of the cusps.

33. The method of claim 1 further comprising providing a sewing ring operably coupled to said bioprosthetic valve, said sewing ring having a surface constructed and arranged to reduce neointimal proliferation.

34. The valve prosthesis of claim 14 further comprising a sewing ring operably coupled to said bioprosthetic valve, said sewing ring having a surface constructed and arranged to reduce neointimal proliferation and pannus formation.
